(19) United States
(12) Patent Application Publication
(10) Pub. No.: US 2006/0089510 A1
(43) Pub. Date: Apr. 27, 2006

(54) Novel compositions of monomers, oligomers and polymers and methods for making the same

(76) Inventors: Gangadhar Panambur, Honolulu, HI (US); Rameshkumar Chellappan, Honolulu, HI (US); Vishnu Vardhan Reddy Karnati, Honolulu, HI (US)

Correspondence Address: DECHERT LLP, P.O. BOX 10004, PALO ALTO, CA, 94303, US

(21) Appl. No.: 10/974,549

(22) Filed: Oct. 26, 2004

(51) Int. Cl. C07C 61/08

(52) U.S. Cl. 562/400

(57) ABSTRACT

A novel monomer composition is described. The novel monomer composition includes at least one property imparting unit. The property imparting unit has at least one member selected from the group consisting of conductivity imparting unit, stability imparting unit and any combinations thereof. The novel monomer may further include a delinking agent, which is attached to at least one of said property imparting unit. The delinking agent includes at least one member selected from the group consisting of C—C bond, CH₂, CH₃, CF₂, CF₃, Si, O, S, functional groups, aromatic residues and any combinations thereof.

### FIELD OF THE INVENTION

The present invention relates to novel compositions of monomers, oligomers and polymers and methods of making the same. More particularly, the present invention relates to compositions of monomers, oligomers and polymers with certain desirable properties and methods of making the same. Consequently, polymers of the present are useful as an electrolyte, especially as a fuel cell ionomer.

### BACKGROUND OF THE INVENTION

With the growing need for energy in the presence of limited fossil fuel supply, the demand for environmentally friendly and renewable energy sources is increasing. Fuel cell technology, a promising source of clean energy production, is the leading candidate to meet the growing need for energy. Fuel cells are efficient energy generating devices that are quiet during operation, fuel flexible (i.e., have the potential to use multiple fuel sources), and have co-generative capabilities (i.e., can produce electricity and usable heat, which may ultimately be converted to electricity). Of the various fuel cell types, the proton exchange membrane fuel cell (PEMFC) has the greatest potential. PEMFCs can be used for energy applications spanning the stationary, portable electronic equipment and automotive markets.

At the heart of the PEMFC is a fuel cell membrane (hereinafter “proton exchange membrane”), which separates the anode and cathode compartments of the fuel cell. The proton exchange membrane controls the performance, efficiency, and other major operational characteristics of the fuel cell. As a result, the membrane should be an effective gas separator, effective ion conducting electrolyte, have a high proton conductivity in order to meet the energy demands of the fuel cell, and have a stable structure to support long fuel cell operational lifetimes. Moreover, the material used to form the membrane should be physically and chemically stable enough to allow for different fuel sources and a variety of operational conditions.

Currently, commercial fuel cell membranes are formed from perfluorinated sulfonic acid (PFSA) materials. A commonly known PFSA membrane is Nafion® and is available from DuPont.

Nafion® and other similar perfluorinated membrane materials manufactured by companies such as W. L. Gore and Asahi Glass (described in U.S. Pat. Nos. 6,287,717 and 6,660,818 respectively) show high oxidative stability as well as good performance. Unfortunately, these perfluorinated membrane materials are very expensive to produce and difficult to manufacture, which significantly hinder the economic viability of fuel cells. At the time of this writing, perfluorinated membranes such as Nafion® cost as much as $500 per m².

To overcome the cost limitations, alternative polymer materials have been actively researched. For instance, partially fluorinated polymer structures such as poly(vinyldifluorides) (PVDF) and PTFE grafted polystyrene, hydrocarbon structures such as aliphatic elastomers and aromatic thermoplastics, and non-fluorinated non-hydrocarbon polymer systems like polyphosphazenes and polysiloxanes have been studied. To date, the most promising of the alternative materials have been acid functionalized aromatic thermoplastics.

Aromatic based thermoplastics, such as poly(ether ether ketone) (PEEK), poly(ether ketone) (PEK), poly(sulfone-udel) (PSU), poly(ether sulfone) (PES), and others have performed well as fuel cell membranes due to their low cost and good film forming characteristics. When functionalized with sulfonic acid or ion exchange moieties, these materials can be used as fuel cell membranes, as described in the following publications: U.S. Pat. No. 6,465,136; U.S. Pat. No. 6,790,931; J. Polym. Sci., Part A, 34, 2421 (1996); J. Appl. Polym. Sci. 61, 1205 (1996); J. Membr. Sci. 139, 211 (1998); Macromolecules 33, 7609 (2000); Electrochem. Acta 46, 2401 (2001); J. Appl. Polym. Sci. 77, 1250 (2000); Electrochem. Syst. 3, 93 (2000); J. Polym. Sci. 70, 477 (1998); Macromolecules 25, 6495 (1992); Solid State Ionics 106, 219 (1998); Solid State Ionics 106, 219-225 (1998); U.S. Patent App 20040028976; and Solid State Ionics 106, 219 (1998).

Most of these polymers are synthesized through a post-modification procedure, in which ion conducting groups are attached to a preformed polymer chain. This procedure suffers from several drawbacks. For example, the ion conducting moieties attach to the chemically activated sites, making such sites more susceptible to attack from other undesirable active chemical species. As another example, this procedure offers no control over the microstructure of the resulting polymer, as the post-modification occurs through the random attachment of ionic moieties to a reactant polymer chain. Additionally, post modification may also lead to undesirable side reactions, such as cross-linking and/or degradation of the polymer. Membranes resulting from these randomly modified polymers typically have higher water uptakes, lower hydrolytic and oxidative stabilities, and poor mechanical properties. Furthermore, these membranes do not enjoy the requisite high performance or long term fuel cell operational stability.

Others have synthesized polymers for use as polymer electrolytes through polymerization routes starting from monomers that were pre-functionalized with ion conducting groups. For example, the publication Guiver et al., Macromolecules 37, 6748-6754 (2004), discusses preparing a series of arylene ether ketones using sodium-6,7 -dihydroxy-2-naphthalene sulfonate as a monomer to introduce sulfonic acid groups onto the polymer backbone. The resulting polymer electrolyte sulfonic acid groups are distributed randomly as single moieties on polymer repeat units and show properties that are similar to post-sulfonated polymer electrolytes. As another example, Yoon et al. (U.S. Patent Publication 20040097695), describes preparing a group of polymer electrolytes with sulfonic acid pendant to the backbone. Although the location of functional groups in such polymer electrolytes can be placed on deactivated sites making the polymer more stable, they still do not have the requisite significant fuel cell performance improvements over post modified polymers. Specifically, the spacing between ion conducting groups is generally evenly distributed throughout the polymer chain, which distribution is not optimal to imparting the highest performance characteristics to the resulting polymer structure.

To circumvent the shortcomings of the randomly distributed ion conducting groups, some have tried to incorporate segments of functionalized and non-functionalized units, to make block copolymers. The synthesis process begins typically by first making oligomers with several ion conducting groups. The resulting oligomers are next polymerized to yield the resulting polymer electrolyte. The resulting polymer electrolyte includes sections rich in ion conducting groups and includes sections that contain no or little ion conducting groups. Aromatic based block copolymers like those described in U.S. Patent Apps. 20040138387, 20040126666 and 20040186262 claim improved performance when used as fuel cell membranes. Unfortunately, these polymers are made from more lengthy and complicated routes. As a result, it is difficult to control the length of the segment and the molecular weight of the ultimately synthesized polymer, making it difficult to synthesize in a reproducible manner a polymer having the desired quality.

What is therefore needed are novel monomer, oligomer and polymer compositions that provide high performance characteristics when used to manufacture polymer electrolytes, without suffering from the above drawback encountered by their conventional counterpart compositions.

### SUMMARY OF THE INVENTION

To achieve the foregoing, the present invention provides a unique polymer structure which has repeat units with clusters of performance imparting units instead of single groups randomly distributed in its structure. The polymer is synthesized from monomers or oligomers which have a high frequency of property imparting units. The result is an improved polymer electrolyte structure which allows for better conductivity and stability characteristics over prior art counterparts.

In one aspect, the present invention provides a novel monomer composition. The novel monomer composition includes at least one property imparting unit. The property imparting unit has at least one member selected from the group consisting of conductivity imparting unit, stability imparting unit and any combinations thereof. The novel monomer may further include a delinking agent, which is attached to at least one of said property imparting unit. The delinking agent includes at least one member selected from the group consisting of C—C bond, CH₂, CH₃, CF₂, CF₃ Si, O, S, functional group, aromatic residues and any combinations thereof.

In other aspects the present invention also describes preferred embodiments of the novel monomer composition, inventive methods of making such novel compositions, inventive polymer composition and inventive methods of making the same.

### BRIEF DESCRIPTION OF THE FIGURES

FIG. 1 shows a fuel cell diagram.

FIG. 2 shows an additional fuel cell diagram.

FIG. 3 shows a monomer or oligomer structure, according to one embodiment of the present invention.

FIG. 4 shows an exemplar aromatic compound, which is a precursor to synthesize inventive monomer or oligomer structures shown in FIG. 2.

FIG. 5 shows an exemplar aromatic compound, which is a precursor to synthesize inventive monomer or oligomer structures shown in FIG. 2.

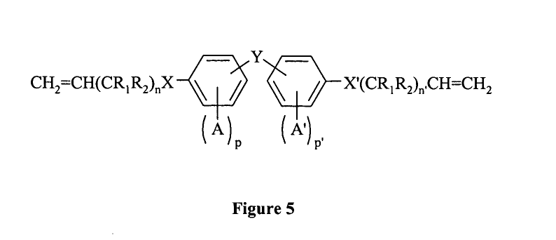

FIG. 6 shows part of a monomer or oligomer structure, which is used to make a polymer structure, according to one embodiment of the present invention.

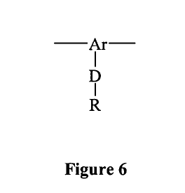

FIG. 7 shows a monomer or an oligomer, which is used to make a polymer structure, according to one embodiment of the present invention.

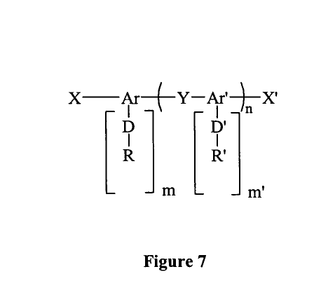

FIG. 8 shows a polymer electrolyte structure, according to one embodiment of the present invention.

FIG. 9 shows a polymer electrolyte structure, according to another embodiment of the present invention.

FIG. 10 shows a polymer electrolyte structure, according to another embodiment of the present invention.

FIG. 11 depicts the Polymer Electrolyte embodiment with at least two repeat units—one repeat unit having two property imparting units and the other with none.

FIG. 12 shows one repeat unit having two property imparting units and the other repeating unit with only one property imparting unit.

### DETAILED DESCRIPTION OF INVENTION

Inventive monomers, oligomers and polymers are ideally suited to produce an electrolyte used in electrochemical devices, such as fuel cells. In one implementation, the present invention is particularly useful for producing a proton exchange membrane in fuel cell applications, as it enjoys better performance and higher stability properties over conventional membranes.

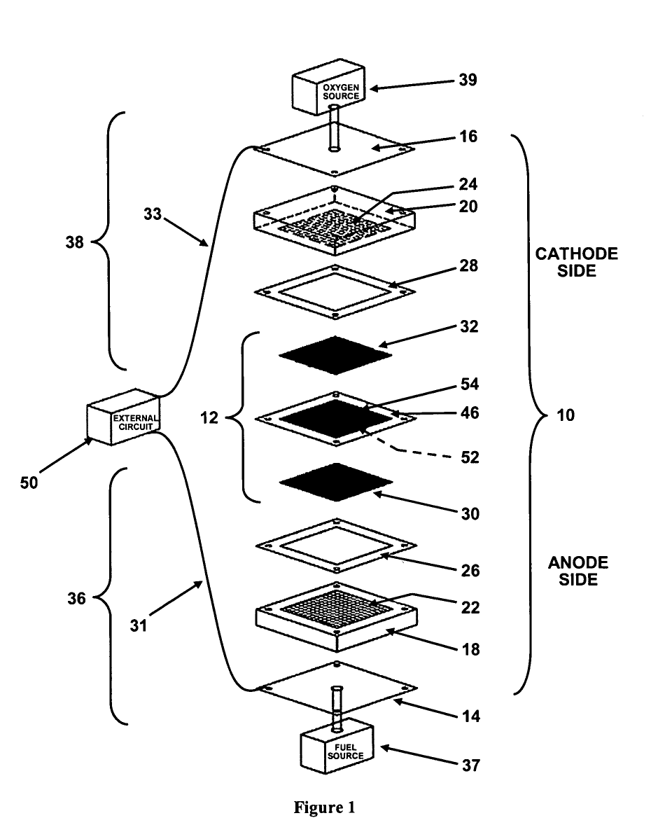

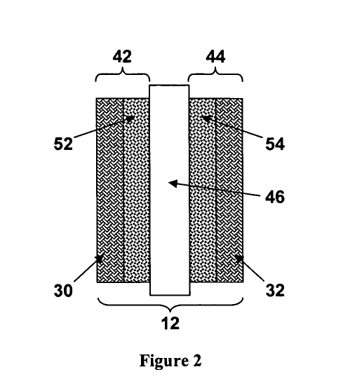

FIG. 1 shows a fuel cell 10 that has incorporated into it an MEA 12, in accordance with one embodiment of the present invention. MEA 12, includes an inventive proton exchange membrane 46, which is shown in greater detail in FIG. 2 and mentioned above. However, it should be noted that the application of inventive membranes are not limited to the fuel cell configuration shown in FIG. 1, rather they can also be effectively employed in conventional fuel cell applications described in U.S. Pat. Nos. 5,248,566 and 5,547,777, which are incorporated by reference herein for all purposes. Furthermore, several fuel cells may be connected in series by conventional techniques to create a fuel cell stack, which contains at least one of the inventive membranes.

As shown in FIG. 1, MEA 12 flanked by anode and cathode structures. On the anode side, fuel cell 10 includes an endplate 14, graphite block or bipolar plate 18 with openings 22 to facilitate gas distribution, gasket 26, and anode current collector 30. On the cathode side, fuel cell 10 similarly includes an endplate 16, graphite block or bipolar plate 20 with openings 24 to facilitate gas distribution, gasket 28, and cathode current collector 32.

Endplates 14 and 16 are connected to external load circuit 50 by leads 31 and 33, respectively. External circuit 50 can be comprised of any conventional electronic device or load such as those described in U.S. Pat. Nos. 5,248,566, 5,272,017, 5,547,777, and 6,387,556, which are incorporated herein by reference for all purposes. The electrical components can be hermetically sealed by techniques well known to those skilled in the art.

During operation, in fuel cell 10 of FIG. 1, fuel from fuel source 37 (e.g., container or ampule) diffuses through the anode and oxygen from an oxygen source 39 (e.g., container, ampule, or air) diffuses to the cathode of the MEA. The chemical reactions at the MEA generate electricity that is transported to the external circuit. Hydrogen fuel cells use hydrogen for fuel and oxygen (either pure or in air) as the oxidant. In direct methanol fuel cells, the fuel is liquid methanol.

Endplates 14 and 16 are made from a relatively dimensionally stable material. Preferably, such material includes one selected from the group consisting of metal and metal alloy. Bipolar plates 18 and 20 are typically made from any conductive material selected from the group consisting of graphite, carbon, metal, and metal alloys. Gaskets, 26 and 28 are typically made of any material selected from the group consisting of Teflon®, fiberglass, silicone, and rubber.

FIG. 2 shows a side-sectional view of MEA 12, which is incorporated into fuel cell 10 of FIG. 1. As shown in this embodiment, MEA 12 includes a proton exchange membrane 46 that is flanked by anode 42 and cathode 44. Each electrode is made of a porous electrode material such as carbon cloth or carbon paper with some type of catalyst dispersion. The proton exchange membrane is at least partially made from any one of the inventive monomers, oligomers and polymers described below. Such monomer compositions and their corresponding molecular structures are described below in great detail.

In accordance with one embodiment of the present invention, proton exchange membrane 46 and at least parts of anode 42 and cathode 44 are derived from the inventive monomer compositions, which has at least one property imparting unit. The term “property imparting unit,” as it is used with respect to this disclosure, refers to a chemical group or moiety, which imparts a desired property to the ultimately formed polymer. Such a desired property of the resulting polymer, in most instances, also proves beneficial to proton exchange membrane 46, anode 42 and cathode 44. The present invention recognizes that a monomer, oligomer or polymer can be synthesized to have a certain property of interest by including in its composition an appropriate property imparting unit. The property imparting unit can be, for example, a conductivity imparting unit, a stability imparting unit, or any combinations thereof.

A conductivity imparting unit can be any unit that imparts the monomer, oligomer or ultimately produced proton exchange membrane 46 (which is also known as the polymer electrolyte), to have a certain desired conductivity. In a preferred embodiment of the present invention, however, a conductivity imparting unit includes any member selected from the group consisting of sulfonic acid, derivatives of sulfonic acid, phosphonic acid, derivatives of phosphonic acid, carboxylic acid, derivatives of carboxylic acid, heterocycles such as imidazole, benzimidazole and pyrazole and any combinations thereof.

A stability imparting unit can be any unit that imparts the monomer, oligomer or the polymer electrolyte to have a certain desired stability. In a preferred embodiment of the present invention, however, a stability imparting unit includes any member selected from the group consisting of crosslinking agents, antioxidizing agents, blocking agents and any combinations thereof. Representative crosslinking agents include at least one member selected from the group consisting of acrylates, methacrylates, alkynes, epoxides, amines, amine derivatives, fumarates, maleates, maliemides and alkenes including allyls, substituted allyls, vinyls and substituted vinyls or any combinations thereof. Representative antioxidizing agents include metal chelating groups, radical absorbing groups, peroxide decomposition groups such as phosphates, phosphate esters, phosphonic acid, derivatives of phosphonic acid, EDTA, any other metal chelating agents and any combinations thereof. Representative blocking agents include at least one member selected from the group consisting of branched hydrocarbon chains, long hydrocarbon chains, branched fluorocarbon chains, long fluorocarbon chains and any combinations thereof.

Both conductivity and stability imparting units may or may not include a delinking agent. In those embodiments where a delinking agent is used, the delinking agent may vary in composition but include at least one member selected from the group consisting of C—C bond, CH₂, CH₃, CF₂, CF₃, Si, O, S, functional groups, aromatic residues and any combinations thereof. In addition to the delinking agent, the conductivity imparting unit includes any member selected from the group consisting of sulfonic acid, derivatives of sulfonic acid, phosphonic acid, derivatives of phosphonic acid, carboxylic acid, derivatives of carboxylic acid, heterocycles such as imidazole, benzimidazole and pyrazole and any combinations thereof. Similarly, in addition to the delinking agent, the stability imparting unit includes any member selected from the abovedescribed group consisting of crosslinking agents, antioxidizing agents, blocking agents and any combinations thereof.

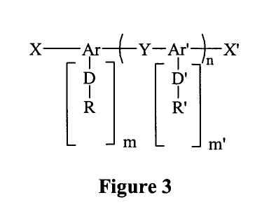

FIG. 3 shows one preferred embodiment of the inventive monomers. Referring to this figure, Ar and Ar′ independently represent an aromatic moiety. In a more preferred embodiment of the present invention, however, Ar and Ar′ independently represent at least one member selected from the group consisting of phenyl, naphthyl, biphenyl, diphenyl ether, diphenylmethane, diphenyldimethylmethane, bisphenone, diphenyl sulfone and most preferably represent a phenyl, biphenyl or naphthyl group.

In FIG. 3, R and R′ independently represent at least one member selected from the group consisting of functional groups, aliphatic groups, aromatic groups and any combinations thereof. In a preferred embodiment of the present invention, however, R and R′ include one member selected from the group consisting of alkyl, alkene, alkyne, crosslinkable group and ion conducting moiety. In a more preferred embodiment of the present invention, the alkyl is a CH₃, sec-butyl or tert-butyl, the alkene is any one of vinyl, substituted vinyls, allyl and substituted allyls, the crosslinkable group is any one of the member selected from the group consisting of acrylates, methacrylates, alkenes, alkynes, epoxides, amines, amine derivatives, fumarates, maleate and maliemides and the ion conducting moiety includes at least one member selected from the group consisting of sulfonic acid, derivatives of sulfonic acid, phosphonic acid, derivatives of phosphonic acid, carboxylic acid, derivatives of carboxylic acid, and heterocycles such as imidazole, benzimidazole, pyrazole. More preferable alkenes include vinyl, allyl, 2-methyl allyl, 2-ethyl allyl, 2-propyl allyl, 2-butyl allyl, 2-pentyl allyl, 2-hexyl allyl, alkyne is acetylene or diacetylene. In a most preferred embodiment of the present invention, the ion conducting moiety is a sulfonic acid or a derivatives of sulfonic acid.

In FIG. 3, D is a chemical structure (also referred to as a “delinking agent” herein) separating Ar and R and similarly, D′ (also known as a delinking agent herein) is a chemical structure separating Ar′ and R′. In accordance with one preferred embodiment of the present invention, D and D′ independently include at least one member selected from a group consisting of C—C bond, CH₂, CH₃, CF₂, CF₃, Si, O, S, functional groups, aromatic residues and any combination thereof. In more preferred embodiments, inventive compositions of the monomer or the oligomer include D and D′ as independently having at least one member selected from the group consisting of C—C bond, CH₂, CH₃, CF₂, CF₃, Si, O and S. D and D′ most preferably independently include at least one member selected from the group consisting of C—C bond, CH₂, CH₃ and O.

In FIG. 3, Y represents a chemical structure separating Ar and Ar′. Y preferably includes at least one member selected from the group consisting of SO₂, CO, O, S, C—C bond, alkanes, fluoroalkanes, aromatic moiety and any combinations thereof. More preferably, Y includes C—C bond, alkanes and fluoroalkanes. Alkane groups in such more preferred embodiments can be represented by the structure CH₂ and are further described in pending U.S. patent application Ser. No 10/851,414. Similarly, fluoroalkanes in such more preferred embodiments can be represented by the structure CF₂ and are also described in the same pending U.S. patent application.

In FIG. 3, X and X′ independently include at least one member selected from the group consisting of F, Cl, Br, NO₂, OH, derivatives of OH, SH, derivatives of SH, COOH, derivatives of COOH, NH₂, derivatives of NH₂ or any combinations thereof. In preferred embodiments of the present invention, however, X and X′ independently include at least one member selected from the group consisting of F, Cl, Br, NO₂, OH, derivatives of OH, SH and derivatives of SH. In more preferred embodiments of the present invention, X and X′ independently include at least one member selected from the group consisting of OH, derivatives of OH, SH and derivatives of SH.

Referring to FIG. 3, the letter “n” is an integer ranging from 0 to 20, but is preferably either 0 or 1. In this figure, the letters “m” and “m′” independently represent an integer value that ranges from 0 to 10, but preferably independently have a value that ranges from 0 to 4, where the sum of “m” and “m′” equals at least 1.

In an exemplar of the most preferred embodiment, the inventive monomers or oligomers include the following structure.

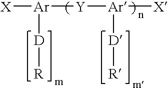

In this embodiment, Ar and Ar′ are phenyl groups. R and R′ include any one selected from the group of methyl, vinyl, allyl, 2-methyl allyl, 2-ethyl allyl, 2-propyl allyl, 2-butyl allyl, 2-pentyl allyl, 2-hexyl allyl, hydroxyl, sulfonic acid or derivatives of sulfonic acid, but includes at least one property imparting group that is an ion conducting moiety. D and D′ independently include at least one member selected from the group consisting of C—C bond, CH₂, CH₃ and O, where at least one species of D and D′ in the inventive monomer represents a chemical separating structure. Y represents any combinations of C—C bond, alkanes and fluoroalkanes, but the number of carbon atoms between Ar and Ar′ should be no greater than 12. X and X′ includes at least one member selected from the group consisting of OH, derivatives of OH, SH or derivatives of SH. The letter “n” is equal to 0 or 1. The letters “m” and “m′” are independent values, but equal to at least 1 where the sum of “m” and “m′” is at least 2.

The inventive monomers or oligomers described above allow for the incorporation of property imparting groups in a clustered, high frequency orientation. Incorporating such a monomer in a polymer repeat unit lends the resulting polymer advantageous characteristics. By way of example, it has been observed that by increasing the number of ionic exchange sites per polymer repeat unit improves the overall conductivity and performance of the resulting ion exchange membrane. Additionally, increasing the amount of crosslinking units per polymer repeat unit increases overall polymer strength and hydrolytic stability compared to its single, randomly distributed counterpart. The inventive polymers, thus, allows for block copolymer performance with a much more simple assembly process.

Accordingly, the present invention also provides such inventive polymer compositions for making a polymer electrolyte, which is shown as proton exchange membrane 46 in FIG. 1. In accordance with one embodiment of the present invention, the polymer electrolyte is made from the inventive monomer compositions described herein. In this embodiment, the inventive polymer electrolyte compositions include at least one type of polymer repeat unit which includes more than one property imparting unit as described above. It is important to note, however, that the property imparting unit of a resulting polymer repeat unit need not have the same structure and property as its component monomer's property imparting unit.

The property imparting units in the polymer electrolyte include at least one member selected from the group consisting of conductivity imparting units, stability imparting units and any combinations thereof. Alternative embodiments of the inventive polymer electrolyte compositions include at least one inventive polymer repeat unit and at least one delinking agent, which is attached as part of the property imparting unit. Preferred embodiments of the delinking agent in the polymer electrolyte compositions have a structure, which is consistent with the delinking agent structure described with respect to the inventive monomers and, therefore, include at least one member selected from the group consisting of C—C bond, CH₂, CH₃, CF₂, CF₃, Si, O, functional groups, aromatic residues and any combinations thereof. In such alternative embodiments, which include delinking agents, conductivity imparting units and stability imparting units have the same structure as described in the corresponding embodiments of the inventive monomers, which also employ delinking agents.

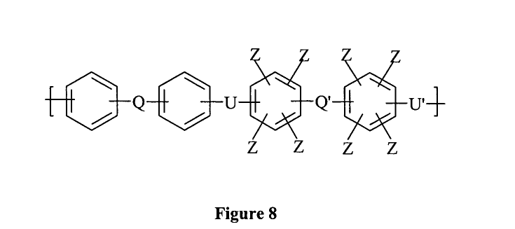

FIG. 8 shows a polymer repeat unit structure found in preferred embodiments of the inventive polymer electrolyte compositions. In this figure, Z independently represents a property imparting unit which is described above, and Q includes at least one member selected from the group consisting of SO₂, CO, CF₂ and any combinations thereof. Q′ of FIG. 8 includes at least one member selected from the group consisting of C—C bond, SO₂, CO, CF₂, CH₂, C(CH₃)₂, C(CF₃)₂, Ar, S, O and any combinations thereof. U and U′ of FIG. 8 independently include at least one member selected from the group consisting of S, O and combinations thereof.

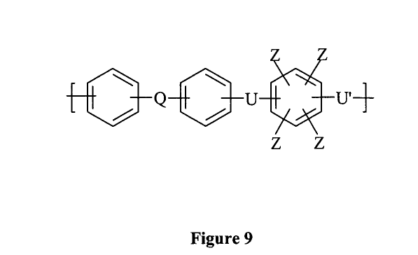

FIG. 9 shows another polymer repeat unit structure, at least one of which is incorporated into other preferred embodiments of the inventive polymer electrolyte compositions. In this figure, Z independently represents the above described property imparting unit. Consequently, Z may include any one of the conductivity imparting unit, a stability imparting unit or any combinations of them. In alternative embodiments of the structure shown in FIG. 9, Z may also include a delinking agent. Q of FIG. 9 is the same as it is in FIG. 8 and, therefore, includes at least one member selected from the group consisting of SO₂, CO, CF₂ and any combinations thereof. As in FIG. 8, U and U′ of FIG. 9 also independently include at least one member selected from the group consisting of S, O and any combinations thereof.

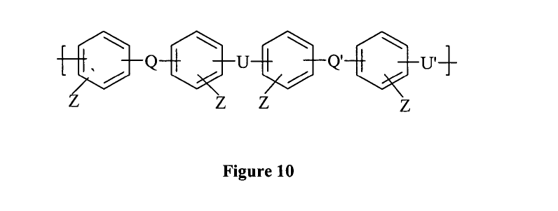

FIG. 10 shows another polymer repeat unit structure, at least one of which is incorporated into other preferred embodiments of the inventive polymer electrolyte compositions. In this figure, Z independently represents the above described property imparting unit. Q, Q′, U and U′ have the same structure and properties as these are described in FIG. 8.

Another polymer repeat unit structure, at least one of which is incorporated into other preferred embodiments of the inventive polymer electrolyte compositions, is shown below. 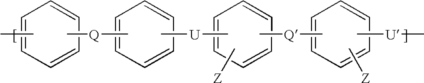
In this figure, Z represents the above described property imparting unit. As shown in FIG. 10, Q, Q′, U and U′ as shown above have the same structure and properties as they are described in FIG. 8.

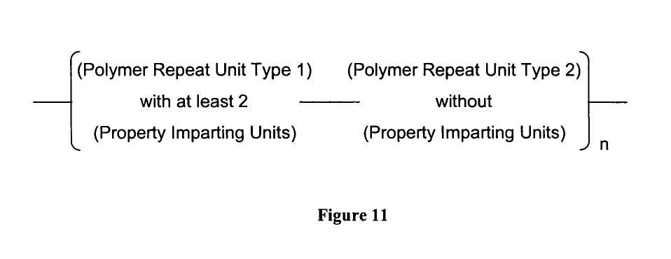

Property imparting units can be distributed throughout the polymer electrolyte structure in many different arrangements, some of which are described below. Preferred embodiments of the inventive polymer electrolyte compositions, for example, include at least two types of polymer repeat units as shown in FIG. 11. According to this figure, a first type of polymer repeat unit includes, and has associated with it more than one property imparting unit. Each of the property imparting units on the first polymer repeat unit include at least one member selected from the group consisting of a conductivity imparting unit, a stability imparting unit and any combinations thereof. The structure and properties of the property imparting unit has been described above in great detail. According to FIG. 11, a second type of polymer repeat unit may include at least one such property imparting unit. Furthermore, the property imparting unit associated with the second type of polymer repeat unit need not be the same as or is independent of the property imparting units associated with the first type of polymer repeat unit. The letter “n” in this figure conveys that the structure of the two polymer repeat units described above is repeated several times over within an inventive polymer electrolyte composition.

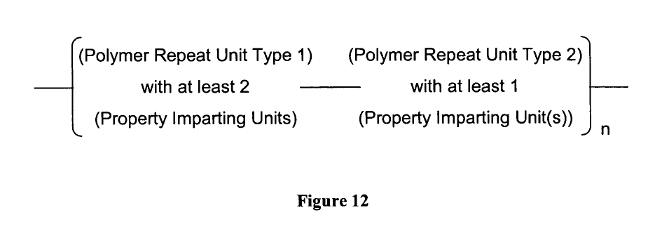

FIG. 12 shows other preferred arrangements of the property imparting units within other polymer electrolyte compositions. According to this figure, the first type of polymer repeat unit has at least two property imparting units and the second type of polymer repeat unit has no property imparting unit.

A yet another preferred arrangement of the property imparting units within the inventive polymer electrolyte compositions is shown below. 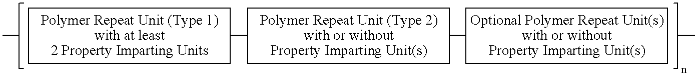
According to this figure, in addition to the two polymer repeat units as shown in each of FIGS. 11 and 12, a third optional polymer repeat unit is found within the polymer electrolyte composition. The third polymer repeat unit may be similar to the second polymer repeat unit in that it may or may not have attached to a property imparting unit. Those skilled in the art will recognize that a polymer electrolyte composition, according to the present invention, may well include more than two types of polymer repeat units.

The present invention also provides techniques for synthesizing the inventive monomer compositions having a property imparting unit, as described above. Synthesis of such inventive monomer compositions depends on the specific property imparting unit that one skilled in the art wishes to impart to the polymer repeat unit and to the ultimately formed polymer electrolyte. For instance, if one wants to impart conductivity through a derivative of sulfonic acid, certain synthesis steps are initiated and performed in a specific order, which would provide a desired monomer composition, having the correct property imparting unit or units.

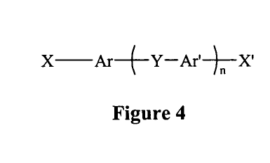

In accordance with one embodiment, the inventive monomer synthesis process is initiated by obtaining the aromatic structure shown in FIG. 4. In this figure, Ar and Ar′ independently include an aromatic moiety. More preferably, Ar and Ar′ independently include an aromatic moiety consisting of phenyl, napthyl, biphenyl, diphenyl ether, diphenylmethane, diphenyldimethylmethane, bisphenone, diphenyl sulfone and any combinations thereof. X and X′ of FIG. 4 independently include at least one member selected from the group consisting of F, Cl, Br, NO₂, OH, SH, derivatives of OH, derivatives of SH, COOH, derivatives of COOH, NH₂, derivatives of NH₂. Y includes at least one member selected from the group consisting of SO₂, CO, O, S, C—C bond, alkanes, fluoroalkanes, aromatic moiety and any combinations thereof. The letter “n” of FIG. 4 is an integer value that ranges from 0 to 20.

Next, one or more side chains are attached to the aromatic structure of FIG. 4. For example, attaching is accomplished by acylating the aromatic compound with n-halo-1-alkanoyl halide to produce an acylated aromatic compound and then reducing the resulting acylated aromatic compound to form a haloalkylated aromatic compound.

After attaching, a subsequent step includes functionalizing at least one of the attached side chains with a property imparting unit. In the above described example, the resulting haloalkylated compound obtained from the attaching step then undergoes reaction in the presence of alkali sulfite to accomplish functionalizing of at least one side chain with a property imparting unit. It is important to note that it is not necessary that the functionalizing step follow the attaching step as described above, in fact, in alternative embodiments it is possible to functionalize the side chain before attaching it to the polymer backbone.

In an alternative embodiment of the inventive synthesis process, the aromatic compound of FIG. 4 undergoes the step of attaching by haloalkylating that aromatic compound with an organometallic reagent and aliphatic dihalide to form a haloalkylated aromatic compound. Next, the step of functionalizing at least one of the side chain includes converting the haloalkylated aromatic compound obtained from the attaching step to sulfonic acid or a derivative of sulfonic acid in the presence of an alkali sulfite.

In another embodiment, the step of attaching delinking side chain to the aromatic groups of the present invention includes using a Claisen rearrangement reaction to attach an allyl or substituted allyl side chain to the aromatic starting compound to form an intermediate aromatic compound. More preferable substituted allyl side chains include 2-methyl allyl, 2-ethyl allyl, 2-propyl allyl, 2-butyl allyl, 2-pentyl allyl and 2-hexyl allyl. Thereafter, the above described attached group is functionalized to form a property imparting group. In one embodiment of the present invention, the attached group is converted to sulfonic acid or a derivative of sulfonic acid units by reacting with alkali hydrogen sulfite.

In a preferred embodiment of the present invention, an acylating procedure begins by obtaining a precursor aromatic compound of the following structure. 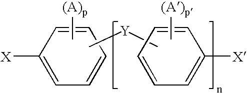 In this structure, X and X′ independently include at least one member selected from the group consisting of F, Cl, Br, NO₂, OH, SH, derivatives of OH, derivatives of SH, COOH, derivatives of COOH, NH₂, derivatives of NH₂. Y includes at least one member selected from the group consisting of SO₂, CO, O, S, C—C bond, alkanes, fluoroalkanes, aromatic moiety and any combinations thereof. The letter “n” is an integer value that ranges from 0 to 20. The letters “A” and “A′” independently represent any substitution on the aromatic moieties and the letters “p” and “p′” independently represent the number of substitutions on aromatic moieties and may vary between 0 and 4.

The precursor aromatic compound is reacted with n-haloalkanoylchloride according to a Friedel Crafts reaction in the presence of a catalyst to make the structure shown below. 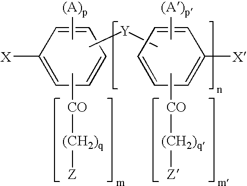
In this structure, X, X′, Y and “n” are described above. Z and Z′ are any member selected from the group consisting of Cl, Br and I. The letters “q” and “q′” independently represent the number of carbon atoms on the side chain and may vary between 1 and 12. The letters “m” and “m′” independently represent the number side chains on aromatic moiety and may vary between 1 and 4. The letters “A” and “A′” independently represent any substitution on the aromatic moieties and the letters “p” and “p′” independently represent the number of substitutions on aromatic moieties and may vary between 0 and 3.

The acylated aromatic compound is reduced using silyl hydride reagent or any other suitable reducing agent to obtain haloalkylated aromatic compound with aliphatic hydrocarbon side chain. The haloalkyl aromatic compound has the general structure shown below. 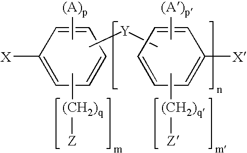
In this structure, X and X′ independently include at least one member selected from the group consisting of F, Cl, Br, NO₂, OH, SH, derivatives of OH, derivatives of SH and NO₂. Y represents at least one compound selected from the group consisting of SO₂, CO, O, S and C—C bond, C(CH₃)₂, C(CF₃)₂, alkane chain and any combinations thereof. The letter “n” is an integer ranging from 0 to 4. Z is any one member selected from the group consisting of Cl, Br and I. The letters “q” and “q′” independently represent the number of carbon atoms on the side chain and may vary between 1 and 13. The letters “m” and “m′” independently represent the number side chains on aromatic moiety and may vary between 1 and 4. The letters “A” and “A′” independently represent any substitution on the aromatic moieties and the letters “p” and “p′” independently represent the number of substitutions on aromatic moieties and may vary between 0 and 3. A preferred embodiment is to incorporate property imparting units by reacting the haloalkylated aromatic compound with alkali sulfite.

A preferred embodiment of making the inventive monomer using the haloalkylation process begins with the compound of the starting structure shown below. 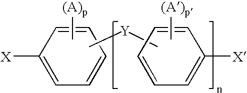
In this structure, X and X′ independently include at least one member selected from the group consisting of F, Cl, Br, NO₂, OH, SH, derivatives of OH, derivatives of SH and NO₂. Y represents at least one compound selected from the group consisting of SO₂, CO, O, S, C—C bond, phenyl, biphenyl, naphthyl, C(CH₃)₂, C(CF₃)₂, alkane chain and any combinations thereof. The letter “n” is an integer ranging from 0 to 4. The letters “A” and “A′” independently represent any substitution on the aromatic moieties and the letters “p” and “p′” independently represent the number of substitutions on aromatic moieties and may vary between 0 and 4.

The aromatic starting compound can be reacted with an organo lithium reagent to lithiate the precursor aromatic starting compound. Next, the lithiated precursor compound is reacted with aliphatic dihalides to form a haloalkylated product. The haloalkylated product may then be functionalized to an ion conducting group by reacting with alkali sulfite.

More preferable embodiments of the present invention include the process of making a monomer using a Claisen rearrangement reaction to attach an allyl, 2-methyl allyl, 2-ethyl allyl, 2-propyl allyl, 2-butyl allyl, 2-pentyl allyl, 2-hexyl allyl, side chain to an aromatic moiety. Preferable embodiments of such a reaction include heating under an inert atmosphere between about 150° C. and about 300° C. any one of aromatic compounds, including allyloxy, 2-methyl allyloxy, 2-ethyl allyloxy, 2-propyl allyloxy, 2-butyl allyloxy, 2-pentyl allyloxy, 2-hexyl allyloxy derivatives and having the structure shown below. 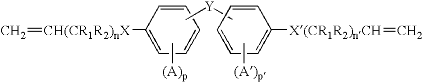
In this structure, X and X′ independently include at least one member selected from the group consisting of 0 and S, Y includes at least one compound selected from the group consisting of SO₂, CO, O, S, C—C bond, C(CH₃)₂, C(CF₃)₂, alkane chain and any combinations thereof. The letters “A” and “A′” independently represent any substitution on the aromatic moieties and the letters “p” and “p′” independently represent the number of substitutions on aromatic moieties and may vary between 0 and 3. R₁ and R₂ independently include at least one member selected from the group consists of H, methyl, ethyl, propyl, butyl, pentyl or hexyl. The letters “n” and “n′” are integers that independently range from 1 to 2.

The aromatic compounds with any one of allyl, 2-methyl allyl, 2-ethyl allyl, 2-propyl allyl, 2-butyl allyl, 2-pentyl allyl, 2-hexyl allyl side chain, have the structure shown below. 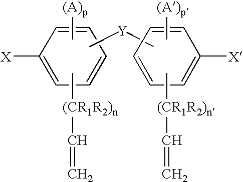
In this structure, X and X′ independently include at least one member selected from the group consisting OH, derivatives of OH, SH or derivatives of SH. Y includes at least one compound selected from the group consisting of SO₂, CO, O, S, C—C bond, C(CH₃)₂, C(CF₃)₂, alkane chain and any combinations thereof. R₁ and R₂ independently include at least one member selected from the group consisting of H, methyl, ethyl, propyl, butyl, pentyl or hexyl. The letter “n” and “n′” are independently integers that range from 1 to 2. The resulting compound is reacted with alkali hydrogen sulfite to prepare the novel monomer of the present invention. The letters “A” and “A′” independently represent any substitution on the aromatic moieties and the letters “p” and “p′” independently represent the number of substitutions on aromatic moieties and may vary between 0 and 3.

Due to the nature of the inventive monomer, it is impossible to fully describe all the synthesis steps to impart specific performance imparting units. However, those skilled in the art will recognize that the synthesis and resulting monomer structures with performance imparting units are novel.

According to the present invention, the polymerization of one or more monomers or oligomers which have at least one property imparting unit made up of either a conductivity imparting unit or a stability imparting unit to make the inventive polymer. Further embodiments of the present invention include synthesizing a polymer electrolyte wherein at least one of the monomers used for polymerization includes the above described delinking agent that is attached to at least one of the property imparting units.

One embodiment of the present invention includes the process of synthesizing a polymer electrolyte wherein the conductivity imparting unit includes a delinking agent and an ion conducting moiety. The ion conducting moiety includes at least one member selected from the group consisting of sulfonic acid, derivatives of sulfonic acid, phosphonic acid, derivatives of phosphonic acid, carboxylic acid, derivatives of carboxylic acid, heterocycles such as imidazole, benzimidazole, pyrazole and any combinations thereof.

Another embodiment of the present invention includes the process of synthesizing a polymer electrolyte containing a stability imparting unit comprised of a delinking agent and a crosslinking agent. Preferred embodiments of the crosslinking agent include at least one member selected from the group consisting of acrylates, methacrylates, alkenes, alkynes, epoxides, amines, amine derivatives, fumarates, maleates, maliemides and any combinations thereof.

Another embodiment of the present invention includes the process of synthesizing a polymer electrolyte containing a stability imparting unit comprising of a delinking agent and an antioxidizing agent. Preferred embodiments of antioxidizing agent include at least one member selected from the group consisting of phosphates, phosphate esters, phosphonic acid, derivatives of phosphonic acid, metal chelating agents and any combinations thereof.

Another embodiment of the present invention includes the process of synthesizing a polymer electrolyte containing a stability imparting unit comprising of a delinking agent and a blocking agent. Preferred embodiments of blocking agent includes at least one member selected from the group consisting of branched hydrocarbon chains, bulky hydrocarbon groups, long hydrocarbon chains, branched fluorocarbon chains, long fluorocarbon chains, bulky fluorocarbon groups and any combinations thereof.

Yet another embodiment of the present invention includes the process of making a polymer electrolyte, comprising polymerizing one or more monomers or oligomers to make the inventive polymer electrolyte where at least one or more monomer or at least one oligomer includes the structure shown below. 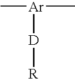
In this structure, Ar represents an aromatic moiety. D is a chemical structure that separates R from Ar, and more preferably includes at least one member selected from a group consisting of C—C bond, CH₂, CH₃, CF₂, CF₃, Si, O, S, functional groups, aromatic residues and any combinations thereof. R represents at least one member selected from the group consisting of functional groups, aliphatic groups and aromatic groups and any combinations thereof. More preferable functional groups of R include ion conducting and cross-linking groups.

Another embodiment of the present invention includes the process of making a polymer electrolyte, comprising polymerizing one or more monomers or oligomers to make an inventive polymer electrolyte, where at least one monomer or at least one oligomer has the structure shown below. 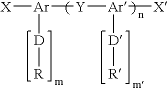
In this structure, where Ar and Ar′ independently include an aromatic moiety, but more preferably include at least one member selected from the group consisting of phenyl, naphthyl, biphenyl, diphenyl ether, diphenylmethane, diphenyldimethylmethane, bisphenone, diphenyl sulfone, and most preferably include phenyl groups. R represents at least one member selected from the group consisting of functional groups, aliphatic groups, aromatic groups and any combinations thereof, but more preferably includes at least one member selected from the group consisting of alkyl, alkene, alkyne, crosslinking agent and any ion conducting moiety. Most preferable alkyl groups are of the CH₃, sec-butyl, tert-butyl type. Most preferable alkenes are allyl, 2-methyl allyl, 2-ethyl allyl, 2-propyl allyl, 2-butyl allyl, 2-pentyl allyl, or 2-hexyl allyl. Most preferable alkynes are acetylene or diacetylene. Most preferable crosslinking agents are acrylates, methacrylates, alkenes, alkynes, epoxides, amines, amine derivatives, fumarates, maleates and maliemides. Preferable ion conducting moieties are sulfonic acid, derivatives of sulfonic acid, phosphonic acid, derivatives of phosphonic acid, carboxylic acid, derivatives of carboxylic acid and heterocycles such as imidazole, benzimidazole, pyrazole and any combinations thereof. Most preferable ion conducting moieties are sulfonic acid and derivatives of sulfonic acid.

D represents a chemical structure separating Ar and R and D′ is a chemical structure separating Ar′ and R′. More preferable embodiments of D and D′ include at least one member selected from the group consisting of C—C bond, CH₂, CH₃, CF₂, CF₃, Si, O, S, functional groups, aromatic residues and any combinations thereof. Even more preferred embodiments of D and D′ include at least one member selected from the group consisting of C—C bond, CH₂, CH₃, CF₂, CF₃, Si, O and S. Y includes a C—C bond, alkanes, fluoroalkanes and any combinations thereof. The most preferable embodiments of D and D′ include at least one member selected from the group consisting of C—C bond, CH₂, CH₃ and O.

Y is a chemical structure separating Ar and Ar′, but more preferably includes at least one member selected from the group consisting of SO₂, CO, O, S, C—C bond, alkanes, fluoroalkanes, aromatic moiety and any combinations thereof.

X and X′ independently include at least one member selected from the group consisting of F, Cl, Br, NO₂, OH, SH, derivatives of OH, derivatives of SH, COOH, derivatives of COOH, NH₂, derivatives of NH₂, but more preferably include at least one member selected from the group consisting of F, Cl, Br, NO₂, OH, SH, derivatives of OH, and derivatives of SH, and most preferably include at least one member selected from the group consisting of OH, SH, derivatives of OH, and derivatives of SH.

The letter “n” is an integer ranging from 0 to 20, but more preferably is 0 or 1. The letters “m” and “m′” independently are integers ranging from 0 to 10 where the sum of “m” and “m′” equals at least 1. More preferable values for “m” and “m′” range from 0 to 4 where the sum of “m” and “m′” equals at least 1.

In yet another embodiment of the present invention, the polymer is made by a nucleophilic polycondensation reaction. Generally, in this embodiment, polymers are synthesized under a dry, inert atmosphere. The above described monomer and oligomer components are dispersed in an aprotic solvent. Typical solvents include, but are not limited to N,N-dimethylformamide (DMF), N,N-dimethylacetamide (DMAc), N-methyl-2-pyrrolidinone (NMP), dimethyl sulfoxide (DMSO), and diphenyl sulfoxide (DPSO) but are more preferably NMP and DMSO. Additionally, an azeotropic component is added to facilitate the removal of water formed as a byproduct from the solution. Typical azeotropic components include at least one member selected from the group consisting of toluene, benzene and xylene. More preferably, however, the azeotrope component includes toluene and benzene. To facilitate the reaction, an inorganic base may be added. The inorganic base includes at least one member selected from the group consisting of potassium carbonate, sodium carbonate, sodium hydroxide, potassium hydroxide and sodium hydride. Preferably, however, the inorganic base includes potassium carbonate. The molar ratio of the inorganic base varies between about 0.75:1 and about 2.5:1, preferably, however, it varies between about 1:1 and about 1.25:1. Reaction temperatures typically range from about 100° C. to about 350° C., but more preferably range from between about 130° C. and about 220° C. Reasonable reaction times range from about 2 to about 72 hours, but more preferably range from between about 5 and about 24 hours. The reaction description describes examples of how the inventive polymers are synthesized. However, those skilled in the art should realize that other mechanisms and reaction parameters may be used to generate the desired polymers that incorporate the inventive monomers or polymer repeat units.

Polymer electrolytes of the present invention made from the disclosed inventive monomers or oligomers show superior properties over conventional polymers. For example, the polymer structures according to the present invention enjoy higher conductivities and higher water stabilities compared to their conventional counterparts. Furthermore, the inventive polymers are more oxidative stable than their conventional counterparts and, therefore, improving fuel cell lifetime. Table 1 below highlights an inventive polymer with at least two ion conducting groups per repeat unit and a conventional counterpart, which has mostly one ion conducting group per polymer repeat unit.

TABLE 1

| | Approximate | | |
|---|---|---|---|
| | Conductivity at | Approximate Water | Approximate |
| | 80° C. and non | Uptake (% after 24 | Linear Expansion |
| | Saturated RH % | hours in H₂O at | (% after 24 hours |
| | (S/cm) | 90° C.) | in H₂O at 90° C.) |
| Inventive | 0.053 | 32% | 9% |
| Polymer | | | |
| (Example 8) | | | |
| Conventional | 0.032 | 57% | 18% |
| Polymer | | | |

It is believed that the improved performance of the inventive polymer is most likely due to the configuration of the polymer which is enhanced through the use of the inventive monomer and the clustering of the property imparting units. Polymer characteristics can also be enhanced by incorporating more than one stability imparting unit per polymer repeat unit. For instance, inventive polymers enjoy lower water uptakes and higher conductivities compared to their conventional counterparts, which have at least two crosslinking units per polymer repeat unit. Table 2 highlights these differences below.

TABLE 2

| | Approximate | | |
|---|---|---|---|
| | Conductivity at | | Approximate |
| | 80° C. and non | Approximate | Water Uptake (% |
| | Saturated RH % | Mechanical | after 24 hours in |
| | (S/cm) | Strength (kpsi) | H₂O at 90° C.) |
| Inventive | 0.083 | 3.513 | 32% |
| Polymer | | | |
| (Example 9) | | | |
| Conventional | 0.075 | 3.815 | 41% |
| Polymer | | | |
| (Example 11) | | | |

The present invention will be described in greater detail with reference to the following examples. These examples are for illustrative purposes only and do not in any way limit the scope of the present invention.

### EXAMPLE 1

This example describes the synthesis of an inventive monomer composition which contains two crosslinkable side chain allyl groups. The synthesis of this inventive monomer involves the following steps.

Step 1 involves the synthesis of 4,4′-diallyloxy biphenyl. About 186 g (1 mol) of 4,4′-biphenol was dissolved in about 1.5 L of ethanol in a round bottom flask. To this solution about 104 g (2.6 mol) of sodium hydroxide was added. The resulting solution was heated to about 70° C. while stirring. Subsequently, about 220 mL (2.6 mol) of allyl bromide was added drop wise over a period of about 30 minutes. Thereafter, the reaction mixture was refluxed at about 80° C. for about 6 hours. Afterwards, the mixture was cooled to about 0° C. and filtered. The filtered product was further washed with about 1 liter of water and dried in an oven at about 80° C. for about 12 hours.

Step 2 involves synthesis of 3,3′-diallyl-4,4′-biphenol. Approximately 239.4 g (0.9 mol) of 4,4′-diallyloxy biphenyl was loaded in a vacuum trap equipped with magnetic stir bar and heating element. High vacuum was applied and the compound was slowly heated to about 250° C. The reaction mixture was held at this temperature while keeping the vacuum on. After about 30 minutes, the reaction mixture was cooled to room temperature. The product was isolated and purified by distillation under vacuum at about 260° C.

### EXAMPLE 2

This example describes the synthesis of another inventive monomer which contains one crosslinkable side chain allyl group. The synthesis of the inventive monomer involves the steps described below.

Step 1 involves synthesis of 4-allyloxy-4′-hydroxy biphenyl. Approximately 186 g (1 mol) of 4,4′-biphenol was dissolved in a round bottom flask containing about 0.5 L of water. The resulting solution was stirred while about 104 g (2.6 mol) of sodium hydroxide was added. After all the sodium hydroxide was dissolved, the solution was slowly heated to about 70° C. Subsequently, about 110 mL (1.3 mol) of allyl bromide was added drop wise. After complete addition of the allyl bromide the reaction mixture was heated for another about 2 hours at about 70° C.. Next, the product was separated by filtration and purified by washing with about 1 liter of water before drying.

Step 2 involves synthesis of 3-allyl-4,4′-biphenol. About 239.4 g (0.9 mol) of 4-allyloxy-4′-hydroxy biphenyl was loaded in a vacuum tube equipped with magnetic stir bar and heating element. High vacuum was applied to the vacuum tube and the compound was slowly heated to about 250° C. After about 30 minutes at about 250° C., the reaction mixture was cooled to room temperature. The solid product was recovered and further purified by distillation under vacuum at about 260° C.

### EXAMPLE 3

This example describes the synthesis of an inventive monomer composition, which contains two ion conducting propylsulfonate groups.

Step 1 involves the synthesis of 3,3′-diallyl-4,4′-diacetoxy biphenyl. Approximately 203.49 g (0.765 mol) of 3,3′-diallyl-4,4′-biphenol was dissolved in about 1 liter of methylene chloride. To the resulting solution about 10 mg of 4-dimethylaminopyridine (DMAP) and about 61.87 mL (0.765 mol) of pyridine were added. Nitrogen gas was then bubbled through the solution and the reaction mixture was slowly cooled to about 0° C. while stirring. Next, about 173.55 mL (1.83 mol) of acetic anhydride was added dropwise by maintaining the reaction temperature at about 0° C. After complete addition of the acetic anhydride, the reaction mixture was slowly warmed to room temperature. The reaction mixture was then stirred for about three hours. About 500 mL of cold water was then added to the reaction mixture and stirred for an additional hour. The resulting solution was washed with about 3 liters of cold water and dried over Na₂SO₄. Subsequently, the resulting solution was concentrated and recrystallized with hexane.

Step 2 involves the synthesis of 3,3′-di(sodium-3-propyl sulfonate)-4,4′-biphenol) (IUPAC name di sodium salt of 3-[4,4′-dihydroxy-3′(3-sulfo-propyl)-biphenyl-3-yl]-propane-1-sulfonic acid). Approximately 254.36 g (0.726 mol) of 3,3′-diallyl-4,4′-diacetoxy biphenyl was dissolved in about 5 L of methanol and about 462.64 g (4.36 mol) of NaHSO₃ in about 1.25 L of water. Next, about 25.43 g of azobisisobutyronitrile (AIBN) was added to the reaction mixture and refluxed at about 80° C. for about 12 hours in the presence of air bubbling. The methanol was removed and NaHCO₃ (about 100 g) was added and stirred for about 3 hours at room temperature. Finally, the product was precipitated with NaCl, filtered and dried. Crystallization with an isopropanol and water mixture (1:1) yielded the final product.

### EXAMPLE 4

This example describes the synthesis of novel monomer which contains two attached 2-methyl propylsulfonate groups.

Step 1 involves the synthesis of 4,4′-di(2-methyl allyloxy)biphenyl. About 200 g (1.075 mol) of 4,4′-biphenol was dissolved in about 1.5 L of ethanol in a round bottom flask. To this solution, about 111.8 g (2.6 mol) of sodium hydroxide was added. The resulting solution was heated to about 70° C. while stirring. Subsequently, about 273.6 mL (2.6 mol) of 3-chloro-2-methyl propene was added drop wise over a period of about 30 minutes. Thereafter, the reaction mixture was refluxed at about 80° C. for about 6 hours. Next, the mixture was cooled to about 0° C. and filtered. The filtered product was washed with about 1 liter of water and dried in an oven at about 80° C. for about 12 hours.

Step 2 involves the synthesis of 3,3′-(2-methyl allyl)-4,4′-biphenol. About 60 g (0.20 mol) of 4,4′-di(2-methyl allyloxy) biphenyl was loaded in a vacuum trap equipped with magnetic stir bar and heating element. High vacuum was applied and the compound was slowly heated to about 250° C. The reaction mixture was held at this temperature while keeping the vacuum on. After about 30 minutes, the reaction mixture was cooled to room temperature. The product was isolated and further purified by distillation under vacuum at 260° C.

Step 3 involves the synthesis of 3,3′-di(2-methyl allyl)-4,4′-diacetoxy biphenyl. About 53 g (0.18 mol) of 3,3′-di(2-methyl allyl)-4,4′-biphenol was dissolved in about 1 liter of methylene chloride. To the resulting solution, about 10 mg of 4-dimethylaminopyridine (DMAP) and about 14.58 mL (0.18 mol) of pyridine were added. Nitrogen gas was then bubbled through the solution and the reaction mixture was slowly cooled to about 0° C. while stirring. Next, about 37.49 mL (0.396 mol) of acetic anhydride was added dropwise by maintaining the reaction temperature at about 0° C. After complete addition of the acetic anhydride, the reaction mixture was slowly warmed to room temperature. The reaction mixture was then stirred for about three hours. About 500 mL of cold water was then added to the reaction mixture and stirred for an additional hour. The resulting solution was washed with about 3 liters of cold water and dried over Na₂SO₄. Subsequently, the resulting solution was concentrated and recrystallized with hexane.

Step 4 involves the synthesis of 3,3′-di(sodium-2-methyl propylsulfonate)-4,4′-biphenol (IUPAC name di sodium salt of 3-[4,4′-dihydroxy-3′(2-methyl-3-sulfo-propyl)-biphenyl-3-yl]-2-methyl-propane-1-sulfonic acid)

Approximately 38 g (0.1 mol) of 3,3′-di(2-methyl allyl)-4,4′-diacetoxy biphenyl was dissolved in about 800 mL of methanol and about 64.0 g (4.36 mol) of NaHSO₃ in about 200 mL of water. Next, about 3.8 g of azobisisobutyronitrile (AIBN) was added to the reaction mixture and refluxed at about 80° C. for about 12 hours in the presence of air bubbling. The methanol was removed and NaHCO₃ (about 100 g) was added and stirred for about 3 hours at room temperature. Finally, the product was precipitated with NaCl, filtered and dried. Crystallization with an isopropanol and water mixture (1:1) yielded the final product.

### EXAMPLE 5

This example describes the synthesis of novel monomer which contains two ion conducting hexylsulfonate groups. The synthesis of this inventive monomer involves the steps described below.

Step 1 involves the synthesis of 4,4′-dimethoxy biphenyl. Approximately 104 g of NaOH was dissolved in 1.5 liters of water. Subsequently 186 g of 4,4′-biphenol was added slowly and the mixture was warmed to 90° C. while stirring. Afterwards, dimethyl sulfate (571.15 mL, 6 mol) was added drop wise over a period of three hours. The resulting solution was then stirred at 90° C. for 12 hours. The reaction mixture was then cooled and the product isolated by filtering.

Step 2 involves the synthesis of 3,3′-di(6-bromohexyl)-4,4′-dimethoxy biphenyl. Approximately 2 g (9.34 mmol) of 4,4′-dimethoxy biphenyl was dissolved in about 50 mL of methylene chloride. To this solution about 1.246 g (9.34 mmol) AlCl₃ was added. The resulting mixture was then cooled to about 0° C. At this stage, about 1.036 mL (10.28 mmol) of 6-bromohexanoyl chloride was added dropwise while stirring and allowed to mix for about 6 hours. Next, about 3.43 mL (10.28 mmol) of triethyl silane was added drop wise and the reaction mixture continued to mix for about 6 hours. Subsequently, about 1.246 g (9.34 mmol) of AlCl₃ was added slowly to the reaction mixture. The solution was then cooled to about 0° C. and about 1.036 mL (10.28 mmol) of 6-bromohexanoyl chloride was added slowly. The mixture was then warmed to room temperature and stirred for another six hours. Next, a batch of about 3.43 mL (10.28 mmol) triethyl silane was added at about 0° C. and slowly warmed up to room temperature over next 6 hours. The reaction was quenched with water, extracted with methylene chloride (2×50 mL), washed with 100 mL of water and dried over Na₂SO₄. The product was purified by column chromatography.

Step 3 involves the synthesis of 3,3′-di(6-bromohexyl)-4,4′-biphenol. About 2 g (4.38 mmol) of 3,3′-di(6-bromohexyl)-4,4′-dimethoxy biphenyl was dissolved in about 50 mL of methylene chloride. The reaction mixture was then cooled to about −78° C. and subsequently about 0.91 mL (9.64 mmol) of BBr₃ was added drop wise. After the addition of BBr₃ was completed, the solution was warmed to room temperature slowly and subsequently quenched with water (about 10 mL). The product was extracted with ether (2×50 mL) and dried over Na₂SO₄ The product was purified by column chromatography.

Step 4 involves the synthesis of 3,3′-di(6-bromohexyl)-4,4′-dimethoxy methyl biphenyl) (IUPAC name di sodium salt of 6-[4,4′-dihydroxy-3′(6-sulfo-hexyl)-biphenyl-3-yl]-hexane-1-sulfonic acid). Approximately 2.1 g (4.9 mmol) of 3,3′-di(6-bromohexyl)-4,4′-biphenol was dissolved in about 50 mL of methylene chloride. To this solution, about 2.56 mL (14.71 mmol) of N, N-diisopropylethylamine followed by about 0.97 mL (12.7 mmol) of chloromethyl methyl ether was added at about 0° C. After the addition is completed, the mixture is slowly warmed to room temperature and stirred for about 12 hours. The reaction was then quenched with water (about 10 mL) and extracted with methylene chloride (about 2×50 mL). The methylene chloride layer was then dried over Na₂SO₄ The resulting product was purified by column chromatography.

Step 5 involves the synthesis of 3,3′-di(sodium-6-hexylsulfonate)-4,4′-biphenol. About 2 g (3.87 mmol) of 3,3′-di(6-bromohexyl)-4,4′-dimethoxy methyl biphenyl was dissolved in about 10 mL of ethanol. To this solution, a mixture of about 2.44 g (19.38 mmol) of Na₂SO₃ in about 30 mL of water was added. The resulting mixture was then refluxed for over night. The next day, the reaction mixture was cooled to about 0° C. and held for about 2 hours. A white solid separated and was filtered, dried and dissolved in about 20 mL of water. To the resulting solution, about 2 mL of dil. HCl was added and the mixture was stirred at room temperature for about 6 hours before precipitating by adding NaCl. The resulting white compound was filtered, dried and recrystallized with isopropanol and water.

### EXAMPLE 6

This example describes the synthesis of an inventive monomer composition, containing four propylsulfonate groups attached as a side chain. The inventive monomer synthesis involved the steps described below.

Step 1 involves the synthesis of 3,3′-diallyl-4,4′-diallyloxy biphenyl. About 194 g (0.729 mol) of 3,3′-diallyl-4,4′-biphenol was dissolved in 1.5 L of ethanol. To this solution 75.85 g (1.896 mol) of NaOH was added and the temperature was slowly raised to 70° C. At this stage about 160.35 mL (1.896 mol) of allyl bromide was added drop wise over 30 minutes and then the reaction mixture was refluxed at 80° C. for 6 hours. Afterwards the reaction mixture cooled to 0° C. and was kept at this temperature for an additional 6 hours. The resulting white solid product was collected by filtering and then washed with 1 L of water and dried in the oven.

Step 2 involves the synthesis of 3,3′,5,5′-tetraallyl-4,4′-biphenol. About 100 g (0.289 mol) of 3,3′-diallyl-4,4′-diallyloxy biphenyl was loaded in a vacuum trap equipped with a magnetic stir bar and heating element. Under vacuum the compound was heated slowly to 250° C. and held for 30 min. Afterwards, the reaction mixture was cooled to room temperature. The product was further purified by distillation.

Step 3 involves the synthesis of 3,3′,5,5′-tetraallyl-4,4′-diacetoxy biphenyl. About 80 g (0.231 mol) of 3,3′,5,5′-tetraallyl-4,4′-biphenol was dissolved in 0.5 L of methylene chloride. The resulting solution was cooled to 0° C. Subsequently, 10 mg of DMAP, 18.7 ml (0.231 mol) of pyridine and 48.08 mL (0.508 mol) of acetic anhydride were added while stirring under nitrogen. The reaction mixture was further stirred for 3 hours at room temperature. Afterwards, 500 mL of water was added and stirred for another hour. The resulting solution was washed with water (3×500 ml), dried over Na₂SO₄, and concentrated. Finally, the product was isolated and purified by crystallization with hexane.

Step 4 involves the synthesis of 3,3′,5,5′-tetra(sodium-3-propyl sulfonate)-4,4′-biphenol. About 15 g (34.88 mmol) of 3,3′,5,5′-tetraallyl-4,4′-diacetoxy biphenyl was dissolved in 300 mL of methanol while stirring and then heated to 80° C. At this stage a mixture of about 44.4 g (418.6 mmol) of NaHSO₃, about 75 mL of water and about 3 g of AIBN were added to the reaction mixture. The resulting reaction mixture was stirred for 12 hours with air bubbling. Afterwards, the methanol was removed by evaporation. Subsequently, about 20 g of NaHCO₃ was added to the reaction mixture and stirred for 3 hours at room temperature. The product was precipitated from the reaction mixture by adding NaCl. The resulting white product was filtered, dried and recrystallized with isopropanol:water (10:1).

### EXAMPLE 7

This example describes the synthesis of an inventive monomer composition, containing a sulfonated aromatic biphenyl side chain. The inventive monomer synthesis involved the steps described below.

Step 1 involves the synthesis of 2-biphenyl-5-methyl-1,4-dimethoxy benzene. Approximately 80 mL of n-butyl lithium (2.5M solution in hexane) was added to a solution of about 27.6 g (200 mmol) of dimethoxybenzene in about 100 mL of THF at room temperature. The reaction mixture was stirred for about 1 hour and then 4-bromo biphenyl (about 23.3 g; 100 mmol) was added. Stirring was continued for about one hour. The reaction mixture was then slowly heated to about 60° C. and stirred at temperature for about 12 hours. Next, the reaction mixture was cooled to room temperature and quenched by saturated aqueous chloride solution. The product was isolated by removing solvent and further purified by column chromatography.

Step 2 involves the synthesis of 2-biphenyl-5-methyl-1,4-hydroquinone. Approximately 22.0 g (75.8 mmol) of biphenyl-5-methyl-1,4-dimethoxy benzene was dissolved in about 50 mL methylene chloride. The resulting solution was cooled to about −78° C. About 17.9 mL (189 mmol) boron tribromide was added dropwise over a period of about one hour. Afterwards, the reaction mixture was allowed to warm to room temperature and stirred for about 12 hours. The reaction was then terminated by quenching with ice water. The final product was isolated by extracting with ether and purified by column chromatography.

Step 3 involves the synthesis of 2-(sodium-4-biphenyl sulfonate)-5-methyl-1,4-hydroquinone. Approximately 5 g (19.0 mmol) of 2-biphenyl-5-methyl-1,4-hydroquinone was dissolved in about 10 mL of chloroform. This solution was cooled to about 0° C. and subsequently about 1.33 mL (19 mmol) of chlorosulfonic acid was added. The reaction mixture was then slowly warmed to room temperature and stirred for about 4 hours. Next, the reaction mixture was poured into ice cooled water and the product was isolated by saturating the aqueous layer with sodium chloride. The product was further purified by crystallization in isopropanol and water (about 5:1 mixture).

### EXAMPLE 8

This example describes the preparation of a preferred embodiment of a polymer, which contains at least one polymer repeat unit that has at least two side chain sulfonic acid groups.

4-fluorophenyl sulfone (about 25.25 g, 0.1 mol) was reacted with 3,3′-bis(sodium-3-propyl sulfonate)-4,4′-biphenol (about 14.23 g, 0.03 mol ) and 4,4′-biphenol (about 13.03 g, 0.07 mol) in the presence of potassium carbonate (about 15.89 g, 0.115 mol) under a dry nitrogen atmosphere in a round bottom flask equipped with nitrogen inlet and a Dean-Stark trap using DMSO (about 300 mL) and benzene. After refluxing/recycling of benzene at about 150° C. for about 4 hours, all the benzene was removed and the heating was continued for about 6 hours at about 160° C. The mixture was cooled and additional DMSO (about 100 mL) was added to the reaction mixture. The viscous solution was poured into a large excess of water in order to obtain a transparent white polymer. The resulting product was washed, filtered and dried.

### EXAMPLE 9

This example describes a preferred embodiment of the inventive polymer electrolyte, which contains two sulfonic acid moeties and two crosslinkable allyl side chains on the same or separate polymer repeat units.

3,3′-disodium sulfonate-4,4′-difluorophenyl sulfone (about 16.04 g, 0.035 mol) was reacted with 4-fluorophenyl sulfone (about 16.53. g, 0.065 mol), 4,4′-biphenol (about 13.96 g. 0.075 mol ) and 3,3′-diallyl-4,4′-biphenol (about 6.65 g, 0.025 mol) is reacted in the presence of potassium carbonate (about 15.89 g, 0.115 mol) under a dry nitrogen atmosphere as explained in Example 8.

### EXAMPLE 10

This example describes another preferred embodiment of the inventive polymer electrolyte, which contains two sulfonic acid moieties and two radical blocking alkyl side chains per polymer repeat unit. 3,3′-disodium sulfonate-4,4′-difluorophenyl sulfone (about 13.75 g, 0.030 mol) was reacted with 4,4′-difluorophenyl sulfone (about 17.80. g, 0.070 mol), 4,4′-biphenol (about 9.3 g. 0.05 mol) and 3,3′-dipropyl-4,4′-biphenol (about 10.4 g, 0.05 mol) is reacted in the presence of potassium carbonate (about 15.89 g, 0.115 mol) under a dry nitrogen atmosphere similar to Example 8.

### EXAMPLE 11

This example describes the synthesis of a conventional polymer electrolyte, which is mentioned in Table 2 and used in this disclosure for comparative purposes. This polymer electrolyte contains one crosslinkable allyl side chain on the same or separate polymer repeat units.

3,3′-disodium sulfonate-4,4′-difluoropheneyl sulfone (about 16.04 g, 0.035 mol) was reacted with 4-difluorophenyl sulfone (about 16.53 g, 0.065 mol), 4,4′-biphenol (about 9.31 g, 0.05 mol ) and 3-diallyl-4,4′-biphenol (about 11.3 g, 0.05 mol) in the presence of potassium carbonate (about 15.89 g, 0.115 mol) under a dry nitrogen atmosphere as explained in Example 8.

Although the present invention is described in terms of fuel cell applications, those skilled in the art will recognize that the inventive structures and techniques described herein can be used for other applications. For example, the inventive monomer can be used to synthesize membranes used in separation process, such as liquid-liquid separation, pervaporation, gas-liquid separation, vapor-liquid separation.

What is claimed is:

1. A monomer composition, comprising at least one property imparting unit, said property imparting unit having at least one member selected from the group consisting of conductivity imparting unit, stability imparting unit and any combinations thereof.

2. The monomer composition of claim 2, said monomer further including a delinking agent attached to at least one of said property imparting unit, wherein said delinking agent includes at least one member selected from the group consisting of C—C bond, CH₂, CH₃, CF₂, CF₃, Si, O, S, functional groups, aromatic residues and any combinations thereof.

3. The monomer composition of claim 2, wherein said conductivity imparting unit includes a delinking agent and an ion conducting moiety, said delinking agent includes at least one member selected from the group consisting of C—C bond, CH₂, CH₃, CF₂, CF₃, Si, O, S, functional groups, aromatic residues and any combinations thereof and said ion conducting moiety includes at least one member selected from the group consisting of sulfonic acid, derivatives of sulfonic acid, phosphonic acid, derivatives of phosphonic acid, carboxylic acid, derivatives of carboxylic acid, heterocycles such as imidazole, benzimidazole, pyrazole and any combinations thereof.

4. The monomer composition of claim 2, wherein said stability imparting unit includes a delinking agent and a cross-linking agent, said delinking agent includes at least one member selected from the group consisting of C—C bond, CH₂, CH₃, CF₂, CF₃, Si, O, S, functional groups, aromatic residues and any combinations thereof and said cross-linking agent includes at least one member selected from the group consisting of acrylates, methacrylates, alkenes, alkynes, epoxides, amines and amine derivatives, fumarates, maleates, maliemides and any combinations thereof.

5. The monomer composition of claim 2, wherein said stability imparting unit includes a delinking agent and an antioxidizing agent, said delinking agent includes at least one member selected from the group consisting of C—C bond, CH₂, CH₃, CF₂, CF₃, Si, O, S, functional groups, aromatic residues and any combinations thereof and said antioxidizing agent includes at least one member selected from the group consisting of phosphates, phosphate esters, phosphonic acid, derivatives of phosphonic acid, metal chelating agents and any combinations thereof.

6. The monomer composition of claim 2, wherein said stability imparting unit includes a delinking agent and a blocking agent, said delinking agent includes at least one member selected from the group consisting of C—C bond, CH₂, CH₃, CF₂, CF₃, Si, O, S, functional groups, aromatic residues and any combinations thereof and said blocking agent includes at least one member selected from the group consisting of branched hydrocarbon chains, long hydrocarbon chains, bulky hydrocarbon groups, branched fluorocarbon chains, long fluorocarbon chains, bulky fluorocarbon groups and any combinations thereof.

7. A monomer, comprising: 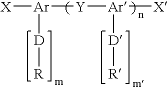
wherein Ar and Ar′ independently include an aromatic moiety,
R represents at least one member selected from the group consisting of functional group, aliphatic group, aromatic group and any combinations thereof,
D is a chemical structure separating Ar and R and D′ is a chemical structure separating Ar′ and R′,
Y is a chemical structure separating Ar and Ar′,
X and X′ independently include at least one member selected from the group consisting of F, Cl, Br, NO₂, OH, SH, derivatives of OH, derivatives of SH, COOH, derivatives of COOH, NH₂, derivatives of NH₂,
n is an integer ranging from 0 to 20,
m and m′ independently are integers ranging from 0 to 10 and a sum of m and m′ equals at least 1.

8. The monomer of claim 7, wherein Ar and Ar′ independently include at least one member selected from the group consisting of phenyl, naphthyl, biphenyl, diphenyl ether, diphenylmethane, diphenyldimethylmethane, bisphenone, diphenyl sulfone.

9. The monomer of claim 7, wherein said functional groups of R and R′ independently include at least one member selected from the group consisting of alkyl, alkene, alkyne, crosslinkable group and any ion conducting moiety.

10. The monomer of claim 9, wherein said alkyl is CH₃, sec-butyl or tert-butyl.

11. The monomer of claim 9, wherein said alkene is any one of vinyl, substituted vinyls, allyl and substituted allyls.

12. The monomer of claim 9, wherein said alkyne is acetylene or diacetylene.

13. The monomer of claim 9, wherein said crosslinkable group is any one of the member selected from the group consisting of acrylates, methacrylates, alkenes, alkynes, epoxides, amines and amine derivatives, fumarates, maleate and maliemides.

14. The monomer of claim 9, wherein said ion conducting moiety includes at least one member selected from the group consisting of sulfonic acid, derivatives of sulfonic acid, phosphonic acid, derivatives of phosphonic acid, carboxylic acid, derivatives of carboxylic acid, and heterocycles such as imidazole, benzimidazole and pyrazole.

15. The monomer of claim 7, wherein D and D′ independently include at least one member selected from a group consisting of C—C bond, CH₂, CH₃, CF₂, CF₃, Si, O, S, functional groups, aromatic residues and any combinations thereof.

16. The monomer of claim 7, wherein Y includes at least one member selected from the group consisting of SO₂, CO, O, S, C—C bond, alkanes, fluoroalkanes, aromatic moiety and any combinations thereof.

17. The monomer of claim 7, wherein X and X′ further independently include at least one member selected from the group consisting of F, Cl, Br, NO₂, OH, SH, derivatives of OH, and derivatives of SH.

18. The monomer of claim 7, wherein Ar and Ar′ include phenyl, R includes any one of sulfonic acid and derivatives of sulfonic acid, D and D′ independently include at least one member selected from the group consisting of C—C bond, CH₂, CH₃, CF₂, CF₃, Si, O and S, Y includes C—C bond, alkanes, fluoroalkanes, X and X′ includes at least one member selected from the group consisting of OH, SH, derivatives of OH, and derivatives of SH, n is either 0 or 1, m and m′ independently are integers ranging from 0 to 4 and said sum of m and m′ equals at least 1.

19. The monomer of claim 18, wherein D and D′ include at least one member selected from the group consisting of C—C bond, CH₃, CH₂ and O.

20. A process of synthesizing a monomer, comprising:
attaching to an aromatic compound one or more side chains;
functionalizing at least one of said one or more side chains with sulfonic acid or with a derivative of sulfonic acid, wherein said aromatic compound having a structure:
X—Ar [symbol] Y—Ar′ [symbol] ₙX′
wherein Ar and Ar′ independently include an aromatic moiety,
X and X′ independently include at least one member selected from the group consisting of F, Cl, Br, NO₂, OH, derivatives of OH, SH, derivatives of SH, COOH, derivatives of COOH, NH₂, derivatives of NH₂,
Y includes at least one member selected from the group consisting of SO₂, CO, O, S, C—C bond, alkanes, fluoroalkanes, aromatic moiety and any combinations thereof, and
n is a integer ranging from 0 to 20.

21. The process of claim 20, wherein said attaching includes:
acylating said aromatic compound with n-halo-1-alkanoyl halide to produce a acylated aromatic compound; and
reducing said acylated aromatic compound to form an haloalkylated aromatic compound.

22. The process of claim 20, wherein said functionalizing includes reacting said haloalkylated aromatic compound with an alkali sulfite.

23. The process of claim 20, wherein said attaching includes using a Claisen rearrangement reaction to attach an allyl or substituted allyl to form an intermediate aromatic compound, and said functionalizing includes converting said intermediate aromatic compound to sulfonic acid or a derivative of sulfonic acid with an alkali bisulfite.

24. A process for making a monomer, comprising:
using a Claisen rearrangement reaction to attach a side chain, which includes any one member selected from the group consisting of allyl, 2-methyl allyl , 2-ethyl allyl, 2-propyl allyl, 2-butyl allyl, 2-pentyl allyl, 2-hexyl allyl to an aromatic compound to form an aromatic compound having said side chains, and
converting said aromatic compound with said side chains to sulfonic acid or a derivative of sulfonic acid in the presence of an alkali bisulfite, wherein said aromatic compound includes the structure: 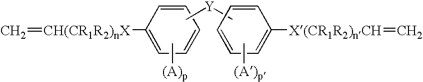
wherein X and X′ independently include at least one member selected from the group consisting of O and S,
Y includes at least compound selected from the group consisting of SO₂, CO, O, S, C—C bond, C(CH₃)₂, C(CF₃)₂, alkane chain and any combinations thereof,
R₁ and R₂ independently include at least one member selected from the group consisting H, methyl, ethyl, propyl, butyl, pentyl and hexyl.
the letters “n” and “n′” are integers that independently range from 1 to 2.
“A” and “A′” independently represent any substitutions on the aromatic moieties.
the letters “p” and “p′” independently represent the number of substitutions on aromatic moieties and may vary between 0 and 3.

25. The process of claim 24, further comprising reacting a 4,4′-biphenol with any one of allyl halides or derivatives of allyl halides to obtain said aromatic compound.

26. The process of claim 24, wherein said Claisen rearrangement involves heating said aromatic compound at a temperature between about 150° C. and about 300° C. under inert atmosphere.
